(19) United States
(12) Plant Patent Application Publication
(10) Pub. No.: US 2002/0129413 P1
(43) Pub. Date: Sep. 12, 2002

(54) Rhodanthe plant named 'Paper Cascade'

(75) Inventor: Esma Salkin, Mount Waverley (AU)

Correspondence Address: Catherine Anne Whealy, 1513 Melody Lane, Roanoke, TX, 76262, US

(73) Assignee: Outback Plants Pty. Ltd., The Patch (AU)

(21) Appl. No.: 09/801,599

(22) Filed: Mar. 9, 2001

(51) Int. Cl. A01H 5/00

(52) U.S. Cl. PLT/263

(57) ABSTRACT

A new and distinct cultivar of Rhodanthe anthemoides named ‘Paper Cascade’, characterized by its cascading plant form; relatively flat inflorescences; and white-colored ray florets.

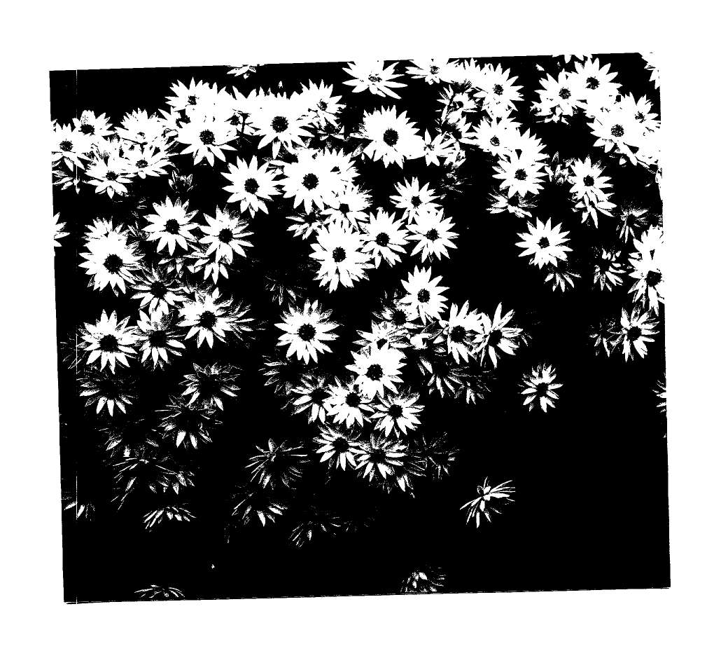

### BACKGROUND OF THE INVENTION

The present invention relates to a new and distinct cultivar of Rhodanthe plant, botanically known as Rhodanthe anthemoides or Helipterum anthemoides and hereinafter referred to by the name ‘Paper Cascade’.

The new Rhodanthe is a product of a planned breeding program conducted by the Inventor in Mount Waverley, Victoria, Australia. The objective of the program is to create and develop new Rhodanthe cultivars.

The new Rhodanthe originated from a cross by the Inventor of two unidentified proprietary selections of Rhodanthe anthemoides, not patented. The new Rhodanthe was discovered and selected by the Inventor as a plant within the progeny of the stated cross in a controlled environment in Mount Waverley, Victoria, Australia in 1987. Plants of the new Rhodanthe differ primarily from the parent selections in growth habit.

Asexual reproduction of the new Rhodanthe by terminal cuttings taken in a controlled environment in Mount Waverley, Victoria, Australia, has shown that the unique features of this new Rhodanthe are stable and reproduced true to type in successive generations.

### SUMMARY OF THE INVENTION

The new Rhodanthe has not been observed under all possible environmental conditions. The phenotype may vary somewhat with variations in environment such as temperature, daylength, light intensity, photoperiod, and water and nutritional status without, however, any variance in genotype.

The following characteristics have been repeatedly observed and are determined to be basic characteristics of ‘Paper Cascade’ and distinguish the new Rhodanthe as a new and distinct cultivar:

1. Cascading plant form.

2. Relatively flat inflorescences.

3. White-colored ray florets.

The new Rhodanthe can be compared to the Rhodanthe anthemoides cultivar Milky Way, not patented. In side-by-side comparisons conducted by the Inventor in Mount Waverley, Victoria, Australia, plants of the new Rhodanthe are more cascading, have flatter inflorescences, and have more pointed ray florets than plants of ‘Milky Way’.

The new Rhodanthe can be compared to the Rhodanthe anthemoides cultivar Paper Star, U.S. Plant Patent application filed concurrently with this application. In side-by-side comparisons conducted by the Inventor in Mount Waverley, Victoria, Australia, plants of the new Rhodanthe differ in bud color, are more cascading, and flower later than plants of ‘Paper Star’.

### BRIEF DESCRIPTION OF THE PHOTOGRAPH

The accompanying colored photograph illustrates the overall appearance of the new cultivar, showing the colors as true as it is reasonably possible to obtain in colored reproductions of this type. Colors in the photograph may differ slightly from the color values cited in the detailed botanical description which accurately describe the colors of the new Rhodanthe. The photograph comprises a side perspective view of typical plants of ‘Paper Cascade’ grown in a container and were about 10 to 14 weeks old.

### DETAILED BOTANICAL DESCRIPTION

The following observations, measurements and values describe plants grown in Mount Waverley, Victoria, Australia, under outdoor conditions with day and night temperatures ranging from 15 to 40° C. and light levels ranging from 5,000 to 9,000 foot candles. Rooted liners of the new Rhodanthe were planted in 15-cm containers and grown for about 10 to 14 weeks. Color references are made to The Royal Horticultural Society Colour Chart except where general terms of ordinary dictionary significance are used.

Botanical Classification: Rhodanthe anthemoides (Helipterum anthemoides) cultivar Paper Cascade.

Parentage:

Male or pollen parent.—Unidentified proprietary selection of Rhodanthe anthemoides, not patented.

Female or seed parent.—Unidentified proprietary selection of Rhodanthe anthemoides, not patented.

Propagation:

Type.—By cuttings.

Time to initiate roots.—Summer: About 8 to 20 days at temperatures of 20 to 25° C. Winter: About 15 to 35 days at temperatures of 20° C.

Root description.—Fine to fibrous, freely branching.

Plant Description:

General appearance.—Decumbent, somewhat rounded and cascading herbaceous plant with linear to narrowly oblanceolate foliage and inflorescences held above and beyond the foliage. Appropriate for 15 pots to 25-cm hanging basket containers.

Plant height.—About 20 to 30 cm.

Plant width.—About 50 to 75 cm.

Branching.—Moderate, removal of terminal apex enhances branching with laterals potentially developing at every node.

Lateral branch description.—Length: About 16 to 39 cm. Width: About 1 to 2 mm. Internode length: About 2 to 7 mm. Fragrance: Chamomile-like when crushed. Texture: Slightly pubescent. Color: 138D.

Foliage description.—Arrangement: Alternate, single. Shape: Linear to narrowly oblanceolate. Apex: Blunt to acute. Base: Decurrent. Margin: Entire, slightly undulate. Length: About 1.4 to 2.7 cm. Width: About 1 to 3 mm. Fragrance: Chamomile-like. Texture: Thin and somewhat fleshy; glabrous or slightly pubescent on both surfaces. Color, both surfaces: 137B.

Flowering Description:

Inflorescence form.—Daisy-type composite inflorescence form. Inflorescences arranged acropetally on a capitulum. Inflorescences close when wet and open when dry.

Inflorescence longevity.—Inflorescences last about 5 to 8 weeks on the plant; about 2 to 3 weeks as a fresh cut flower; and more than 8 weeks as a dried cut flower. Inflorescences persistent.

Natural flowering season.—Natural flowering season is late winter and spring in Australia. Plants flower continuously for about 8 to 10 weeks.

Quantity of inflorescences.—One terminal inflorescence per lateral branch.

Inflorescence aspect.—Flat to slightly cupped.

Inflorescence diameter.—About 2.78 cm.

Inflorescence depth (height).—About 0.8 to 1.1 cm.

Disc diameter.—About 5 to 7 mm.

Fragrance.—Faint sweet honey-like.

Ray florets.—Appearance: Satiny. Texture: Papery. Aspect: Mostly flat. Shape: Narrowly to broadly lanceolate. Margin: Entire. Apex: Acute. Base: Truncate. Length: About 1.3 to 1.6 cm. Width: About 3 to 5 mm. Color: When opening, upper surface: 155D. When opening, lower surface: 59C. Mature, upper surface: 155D; with development, 155C. Mature, lower surface: 155D.

Disc florets.—Shape: Filiform to tubular with two to five lobes. Diameter: About 1 mm. Width: About 1 mm.

Peduncle.—Length: About 1 to 2.5 cm. Aspect: Spreading to erect. Strength: Weak. Texture: Smooth. Color: 138D.

Flower bud.—Shape: Narrowly ovoid. Length: About 1.25 to 1.5 cm. Color: 59C.

Androecium.—Only present on disc florets. Stamen number: One per disc floret. Anther shape: Linear. Amount of pollen: Moderate.

Gynoecium.—Present on both ray and disc florets. Pistil number: One per floret. Style length: About 3 mm. Stigma shape: Bilobed.

Seed.—Seed production is rarely observed; seeds typical of species.

Disease Resistance: Plants of the new Rhodanthe have not been shown to be resistant to pathogens common to Rhodanthe.

It is claimed:

1. A new and distinct cultivar of rhodanthe named ‘Paper Cascade’, as illustrated and described.
